(19) United States
(12) Patent Application Publication
(10) Pub. No.: US 2018/0361089 A1
(43) Pub. Date: Dec. 20, 2018

(54) PORTABLE IN-EXSUFFLATOR

(71) Applicant: SEOILPACIFIC INC., Seoul (KR)

(72) Inventors: Gye Cheol KIM, Gimpo-si, Gyeonggi-do (KR); Chil Hwan KIM, Bucheon-si, Gyeonggi-do (KR)

(73) Assignee: SEOILPACIFIC INC., Seoul (KR)

(21) Appl. No.: 15/778,257

(22) PCT Filed: Oct. 27, 2016
(86) PCT No.: PCT/KR2016/012122

§ 371 Date: May 23, 2018

(30) Foreign Application Priority Data
Jan. 13, 2016 (KR) 10-2016-0004108

(51) Int. Cl. A61M 16/00; A61M 16/08; A61M 16/20

(52) U.S. Cl. CPC A61M 16/0069; A61M 16/0006; A61M 2016/0015; A61M 16/204; A61M 16/024; A61M 16/0875

(57) ABSTRACT

The present invention relates to a portable in-exsufflator, and the objective of the present invention is to provide a portable in-exsufflator for maximally simplifying an air passage so as to minimize a flow loss.

The in-exsufflator comprises: a manifold having a first accommodation part and a second accommodation part, which are formed at an upper part thereof, and an inhalation chamber and an exhalation chamber, which are formed at a lower part thereof; an air pressure generation unit for suctioning and discharging air by means of a fan rotating by a rotational force transmitted from a motor, and having an air suction port connected to the inhalation chamber and an air discharge port connected to the second accommodation part; a direction-switching valve unit coupled to the first accommodation part, and switching a direction so as to allow external air to flow therein and be supplied to respiratory organs or to suction the air from the respiratory organs by using the air pressure generated from the air pressure generation unit; and a high-frequency oscillation wave generation means coupled to the second accommodation part, and generating a high-frequency oscillation wave when the external air having flowed therein is supplied to the respiratory organs or the air is suctioned from the respiratory organs, wherein a plurality of communication ports are formed around the first accommodation part of the manifold such that the communication ports respectively communicate with an external air flow port, the inhalation chamber and the exhalation chamber.

### TECHNICAL FIELD

The present invention relates to a portable in-exsufflator, and more specifically, to a portable in-exsufflator for maximally simplifying an air flow path so as to minimize a flow loss.

### BACKGROUND ART

Coughing is one of the important defensive actions of our body and prevents harmful substances such as gas, bacteria, or the like, or various foreign substances from entering an airway.

Coughing also helps to ensure that the airway is kept clean at all times by allowing suctioned foreign substances or intra-airway secretions to be discharged from the airway.

Accordingly, for example, for patients with impaired coughing function, such as neuromuscular patients with respiratory muscle paralysis, or patients with a restrictive pulmonary disease, pneumonia is caused by foreign substances or secretions block an airway to cause a dyspnea phenomenon.

A cough assist device has been developed to protect a patient from such a risk, and a conventional example thereof has been disclosed in Korean Patent Registration No. 10-1459332 filed by the applicant of the present invention, and titled “Portable cough stimulating device using high frequency vibration wave.”

FIGS. 1 and 2 are views showing an example of a portable in-exsufflator according to the related art.

Referring to FIG. 1, a conventional portable in-exsufflator includes: a manifold 1 including a first accommodation part 1a and a second accommodation part 1b, wherein a plurality of communication holes 1c are formed around the first accommodation part 1a so that the communication holes 1c communicate with an external air flow port 1d, first and second connection ports 1e and 1f communicating with an air pressure generation unit 2, and the second accommodation part 1b communicating with a respiratory flow port 1g connected to respiratory organs of a patient; the air pressure generation unit 2 including an air suction port 2a connected to the first connection port 1e and an air discharge port 2b connected to the second connection port 1f, wherein air is suctioned through the air suction port 2a and the suctioned air is discharged through the air discharge port 2b; a direction-switching valve unit 3 coupled to the first accommodation part 1a, and configured to switch a direction by using the air pressure generated from the air pressure generation unit 2 so as to supply air introduced from the external air flow port 1d to the respiratory organs or to suction the air from the respiratory organ; and a high-frequency oscillation wave generation means 4 coupled to the second accommodation part 1b, and configured to generate a high-frequency oscillation wave when supplying air introduced from the external air flow port 1d to the respiratory organs or suctioning air from the respiratory organs.

Also, the high-frequency oscillation wave generation means 4 includes: a fixed body 4a composed of an inner cylinder 4a-1 in which a flow path F is formed on a central axis and a through hole h is formed in a horizontal direction, and an outer cylinder 4a-2 formed on an outer side of the inner cylinder 4a-1 and having an inner circumferential surface provided with a magnet M; and a moving body 4b installed between the inner cylinder 4a-1 and the magnet M and having a through hole h formed in the horizontal direction and a coil C wound around an outer circumferential surface so that the moving body 4b moves longitudinally by the supply of current, wherein a position sensing magnet 4c is installed on an upper outer circumferential surface of the moving body 4b and a center position sensing sensor 4d is installed on the outer side of the moving body 4b.

When the conventional portable in-exsufflator having the above-described configuration is being operated in a state in which power is turned on and a pressure is set, the air pressure generation unit 2 is driven at a rotational speed corresponding to the set pressure, and the portable in-exsufflator performs an inhalation mode, a pause mode, and an exhalation mode.

In the inhalation mode IN, external air introduced through the external air flow port 1d by a rotator 3a of the direction-switching valve unit 3 passes through the air discharge port 2b through the air suction port 2a of the air pressure generation unit, and the passed external air then passes through the high-frequency oscillation wave generation means 4 once more to supply air to the patient through the respiratory flow port 17.

In the pause mode PA, air introduced into the air suction port 2a of the air pressure generation unit 2 by the rotator 3a of the direction-switching valve unit 3 flows into the air discharge port 2b, and at this point, the airs in the external air flow port 1d and the respiratory flow port 1g are in a state of not flowing into the air pressure generation unit 2.

Also, in the exhalation mode EX, air introduced from the respiratory flow port 1g passes through the high-frequency oscillation wave generation means 4, the air is introduced into the air suction port 2a of the air pressure generation unit 2 by the rotator 3a of the direction-switching valve unit 3 and then discharged to the air discharge port 2b, and the discharged air passes through a flow path formed by the rotator 3a of the direction-switching valve unit 3 to be discharged to the external air flow port 1d.

Here, in the high-frequency oscillation wave generation means 4, the moving body 4b provided between the inner and outer cylinders 4a-1 and 4a-2 of the fixed body 4a moves longitudinally by the supply of current in the inhalation mode IN and the exhalation mode EX. Here, the through hole h formed in the inner cylinder 4a-1 and the through hole h formed in the moving body 4b are repeatedly opened or closed to flow air, thereby forming an oscillation wave. Specifically, the center position sensing sensor 4d and the position sensing magnet 4c accurately sense the position of the through hole h to allow the air to flow, and the frequency and the amplitude are determined by a moving speed and moving distance of the moving body 4b.

However, in the conventional portable in-exsufflator, since the manifold 1 and the air pressure generation unit 2 are horizontally installed, the length of a flow path becomes long and thus a flow loss is great.

Further, in the conventional portable in-exsufflator, fresh external air and exhaled air are likely to be mixed with each other due to the external air introduced into the external air flow port 1d being discharged through the same passage as that of the air exhaled from the patient's respiratory organs, and there is a problem in that the discharged air is directly discharged to the external air flow port 1d, which can cause a great discharge noise.

Also, in the inhalation mode IN and the exhalation mode EX, the oscillation and amplitude control of the high-frequency oscillation wave generation means 4 is controlled by the center position sensing sensor 4d and the position sensing magnet 4c, and thus accurate control is difficult.

Also, in the pause mode, there is a problem in that the flow paths of the respiratory flow port 1g and the external air flow port 1d do not communicate with each other, and therefore the patient is not breathing under atmospheric pressure conditions.

### DISCLOSURE

### Technical Problem

The present invention has been made to solve the above problems of the related art and it is an object of the present invention to provide a portable in-exsufflator which maximally simplifies an air flow path so as to minimize a flow loss.

Further, it is another object of the present invention to provide a portable in-exsufflator which completely separates an inlet through which external air is introduced and a discharge port through which air exhaled from a patient is discharged, and that reduces discharge noise during exhalation.

Further, it is still another object of the present invention to provide a portable in-exsufflator configured to control an oscillation and amplitude of a high-frequency oscillation wave generation means by feeding back a pressure in an inhalation mode IN and exhalation mode EX.

Further, it is yet another object of the present invention to provide a portable in-exsufflator which provides a patient with a comfortable resting function by allowing the patient to breathe under atmospheric pressure conditions.

### Technical Solution

One aspect of the present invention provides a portable in-exsufflator according to the present invention including: a manifold having an upper part in which a first accommodation part and a second accommodation part are formed, and a lower part in which an inhalation chamber and an exhalation chamber are formed; an air pressure generation unit configured to suction and discharge air through a fan rotating by a rotational force transmitted from a motor, and having an air suction port connected to the inhalation chamber and an air discharge port connected to the second accommodation part; a direction-switching valve unit coupled to the first accommodation part, and configured to switch a direction so as to allow external air to flow therein and be supplied to respiratory organs or to suction the air from the respiratory organs by using air pressure generated from the air pressure generation unit; and a high-frequency oscillation wave generation means coupled to the second accommodation part, and generating a high-frequency oscillation wave when external air having flowed therein is supplied to the respiratory organs or the air is suctioned from the respiratory organs, wherein a plurality of communication ports are formed around the first accommodation part of the manifold such that each of the communication ports communicate with an external air flow port, the inhalation chamber and the exhalation chamber.

The communication port communicating with the inhalation chamber and the exhalation chamber may communicate in a vertical direction.

The second accommodation part may have one side at which an air supply chamber is formed so as to communicate thereto, and a discharge port of the air pressure generation unit may be connected to the supply chamber.

The direction-switching valve unit may include a rotator composed of a pair of partition walls configured to selectively open a communication path formed around the first accommodation part and a reversible motor connected to a shaft of the rotator.

The high-frequency oscillation wave generation means may include: a fixed body composed of an inner cylinder in which a flow path is formed on a central axis and a through hole is formed in a horizontal direction, and an outer cylinder formed on an outer side of the inner cylinder and having an inner circumferential surface provided with a magnet; and a moving body installed between the inner cylinder and the magnet and having a through hole formed in the horizontal direction and a coil wound around an outer circumferential surface so that the moving body moves longitudinally by the supply of current.

An oscillation and amplitude may be controlled by the moving body during inhalation and exhalation, and the position of the moving body is controlled by feedback of a pressure of an air flow path.

### Advantageous Effects

In order to accomplish the present invention, a portable in-exsufflator according to the present invention is provided with an air pressure generation unit in a direction perpendicular to the manifold, thereby minimizing the flow loss by maximally simplifying an air flow path.

Also, the portable in-exsufflator according to the present invention forms a flow path configured to introduce external air during inhalation, discharges the air through a central flow path of the high-frequency oscillation wave generation means during exhalation, and blocks air from flowing into an air intake port of the air pressure generation unit during exhalation, so that the inlet configured to introduce the external air and the discharge port configured to discharge the air exhaled from the patient can be completely separated and a discharge noise during the exhalation can be reduced.

Also, the portable in-exsufflator according to the present invention can control an oscillation and amplitude of a high-frequency oscillation wave generation means 40 by feeding back a pressure in an inhalation mode IN and an exhalation mode EX by removing a position sensing magnet provided on an upper part of the conventional moving body and a center position sensing sensor provided on an outside of the magnet.

Further, the portable in-exsufflator according to the present invention communicates flow paths of a respiratory flow port and an external air flow port in a pause mode so that a patient can breathe under atmospheric pressure conditions, thereby providing a comfortable resting function to the patient.

### DESCRIPTION OF DRAWINGS

FIGS. 1 and 2 are views showing an example of a portable in-exsufflator according to the related art.

FIG. 3 is a perspective view of a portable in-exsufflator according to the present invention.

FIG. 4 is a front view of the portable in-exsufflator according to the present invention.

FIG. 5 is a side view of the portable in-exsufflator according to the present invention.

FIG. 6 is a plan view of the portable in-exsufflator according to the present invention.

FIG. 7 is a cross-sectional view taken along line A-A of FIG. 4.

FIG. 8 is a cross-sectional view taken along line B-B of FIG. 6.

FIG. 9 is a plan view and a bottom perspective view of a manifold of the portable in-exsufflator according to the present invention.

FIG. 10 is a cross-sectional view taken along line C-C of FIG. 9.

FIG. 11 is a graph illustrating oscillation waves of the portable in-exsufflator using a high-frequency oscillation wave according to the present invention.

FIG. 12 is a view illustrating an inhalation mode of the portable in-exsufflator using a high-frequency oscillation wave according to the present invention.

FIG. 13 is a view illustrating a pause mode of the portable in-exsufflator using a high-frequency oscillation wave according to the present invention.

FIG. 14 is a view illustrating an exhalation mode of the portable in-exsufflator using a high-frequency oscillation wave according to the present invention.

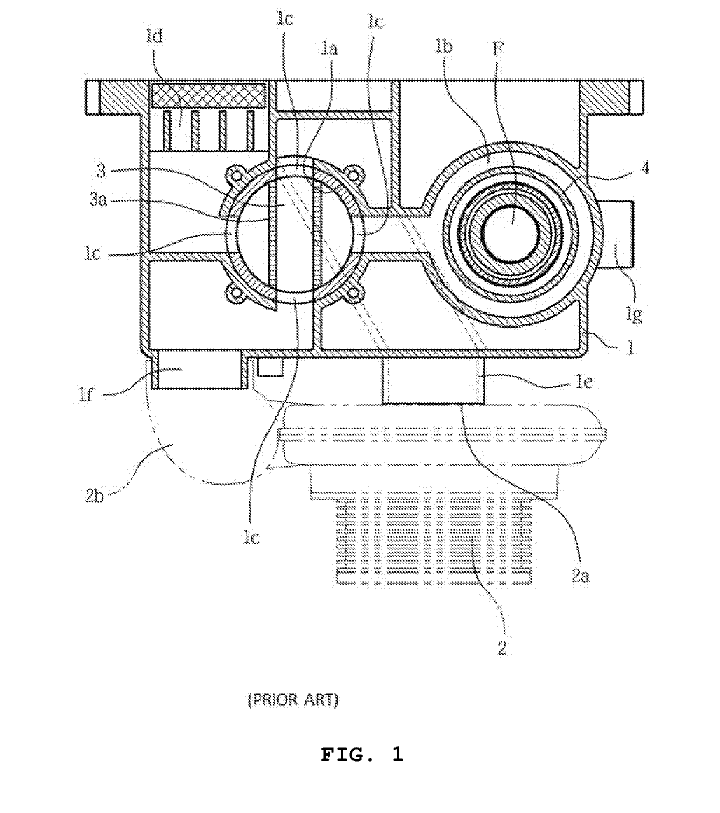

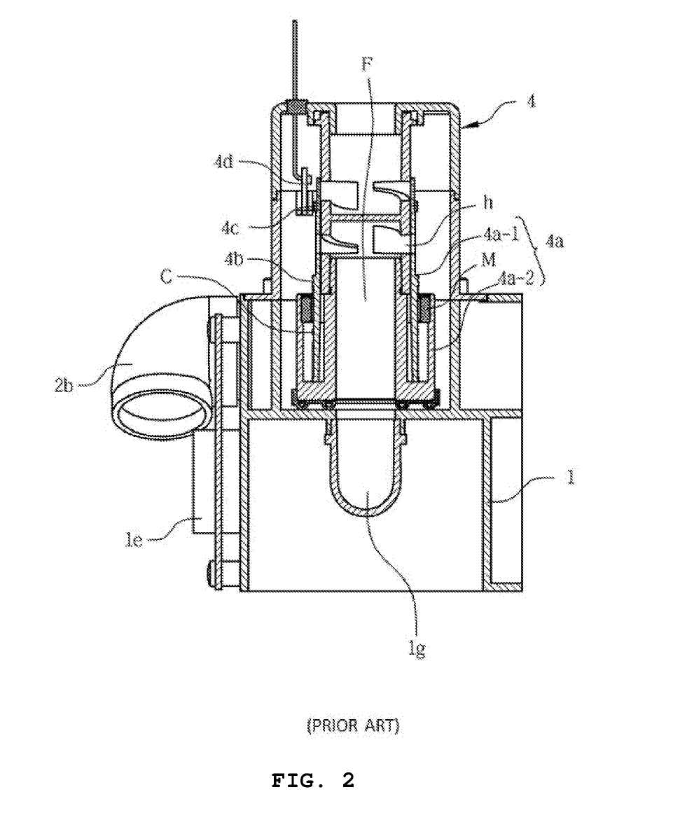

### MODES OF THE INVENTION

Hereinafter, the structure and function of the embodiment of the present invention will be described with reference to the accompanying drawings.

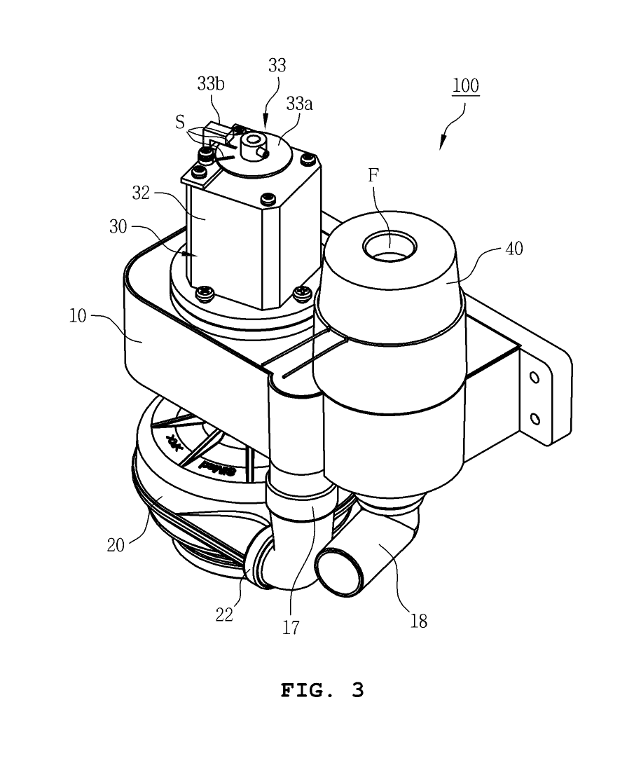

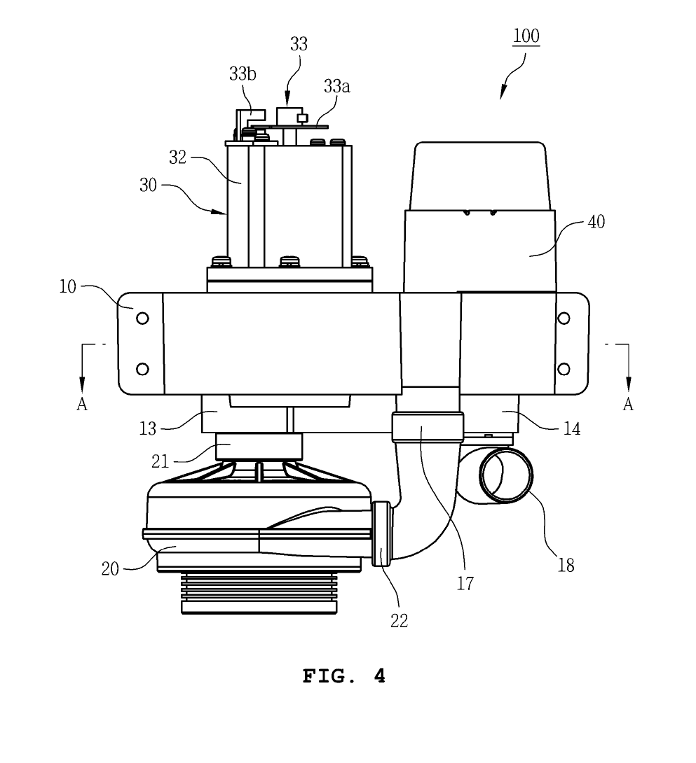

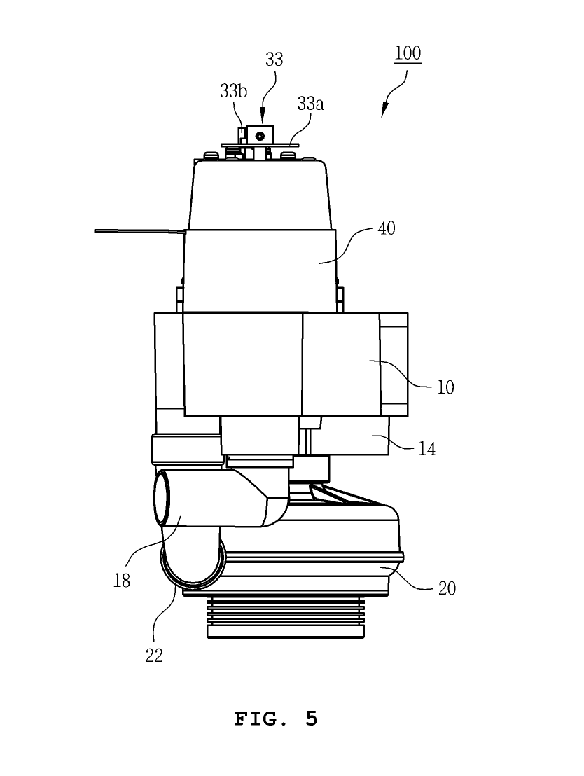

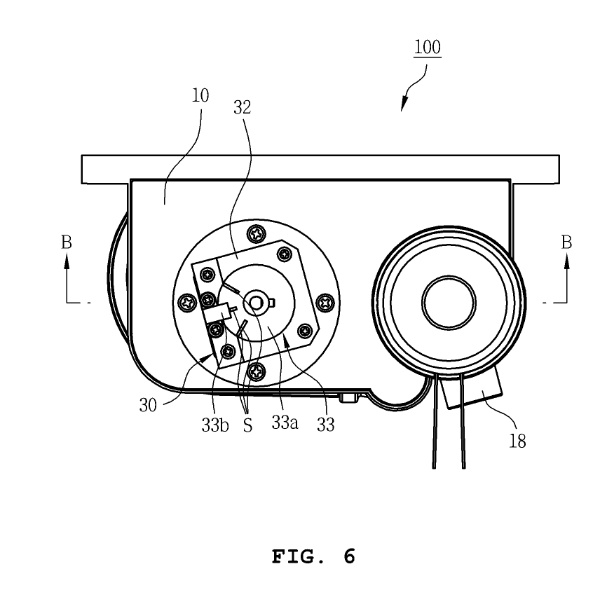

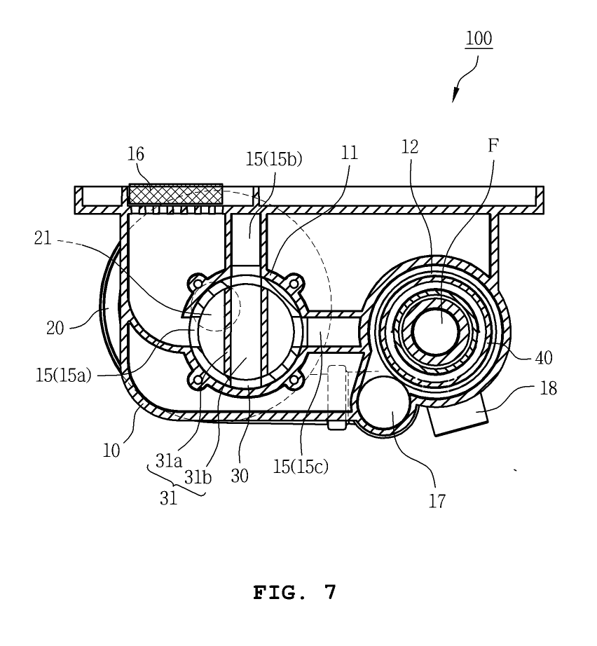

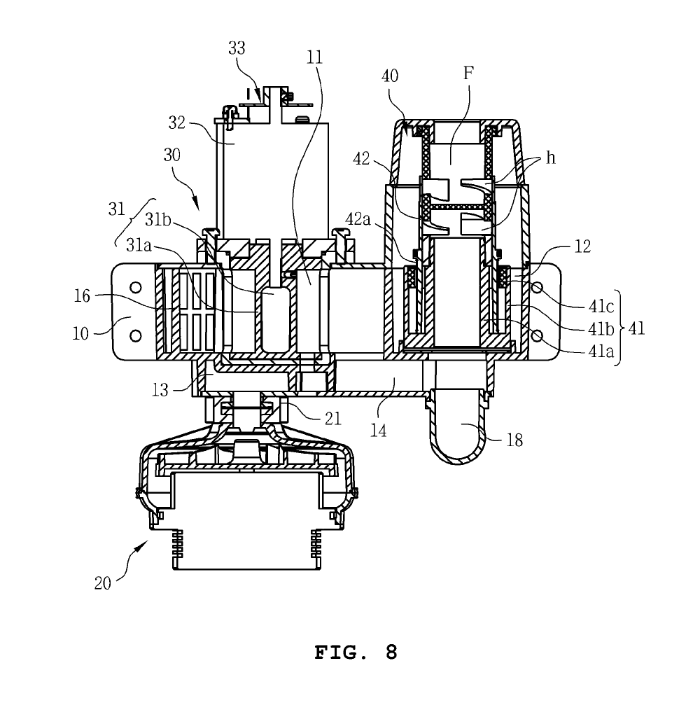

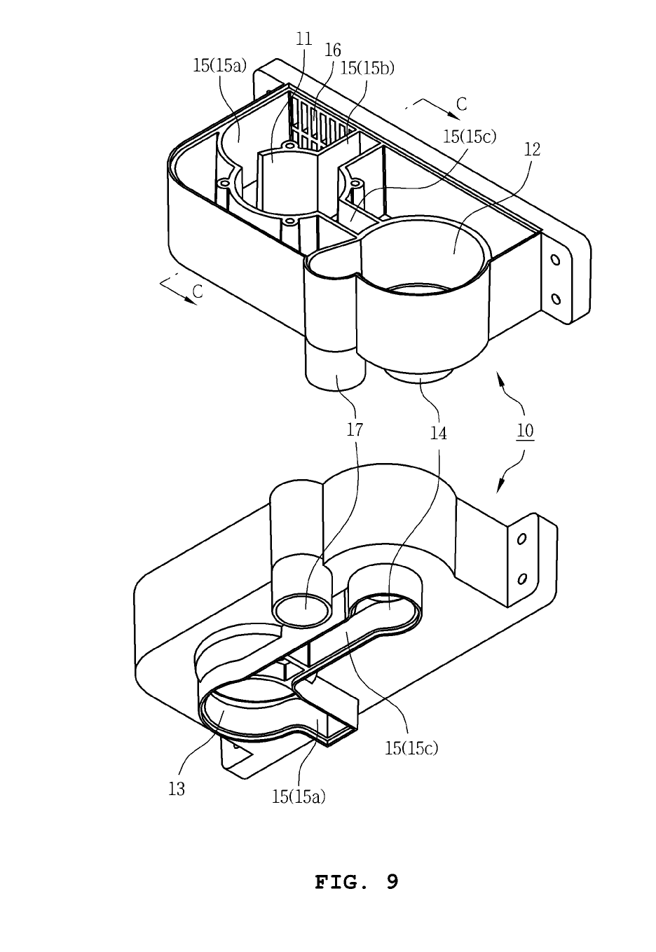

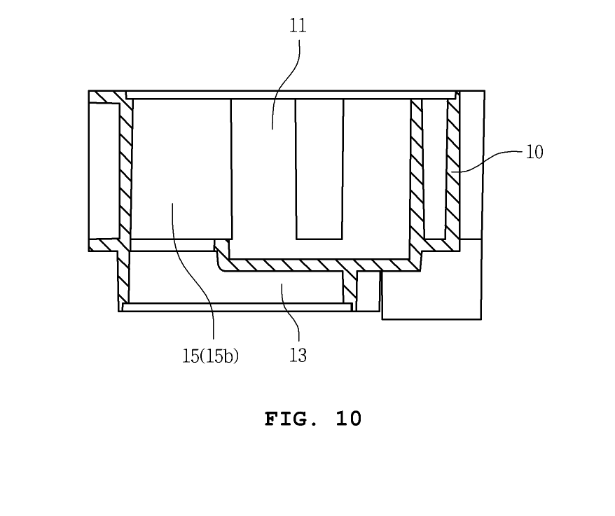

Referring to the FIGS. 3 to 10, a portable in-exsufflator 100 according to the present invention includes a manifold 10, an air pressure generation unit 20, a direction-switching valve unit 30, and a high-frequency oscillation wave generation means 40.

The manifold 10 has a first accommodation part 11 and a second accommodation part 12 formed at an upper part thereof and an inhalation chamber 13 and an exhalation chamber 14 formed at a lower part thereof.

Also, a plurality of communication ports 15 are formed around the first accommodation part 11 of the manifold 10, and each of the communication ports 15 communicates with an external air flow port 16, an inhalation chamber 13, and an exhalation chamber 14.

Also, the communication ports 15 communicating with the inhalation chamber 13 and the exhalation chamber 14 are communicated in a vertical direction, so that the structure of the flow path may be simplified in comparison with an existing one.

Also, the second accommodation part 12 has one side at which an air supply chamber 17 is formed so as to communicate therewith, and an air discharge port 22 of the air pressure generation unit 20, which will be described later, is connected to the air supply chamber 17.

Also, the second accommodation part 12 communicates with a respiratory flow port 18 connected to respiratory organs of a patient.

Also, a filter is installed in the external air flow port 16 so that foreign substances in external air may be filtered out.

The respiratory flow port 18 is not shown in the diagram but is connected to a mask hose configured to be in close contact with a patient's mouth via a bacterial filter through a patient port.

The air pressure generation unit 20 suctions and discharges air through a fan rotating by a rotational force transmitted from a motor. That is, an air suction port 21 is connected to the inhalation chamber 13 and an air discharge port 22 is connected to the second accommodation part 12 so that the air is suctioned through the air suction port 21 and the suctioned air is discharged through the air discharge port 22.

The direction-switching valve unit 30 is coupled to the first accommodation part 11 and uses an air pressure generated from the air pressure generation unit 20 to supply the air introduced from the external air flow port 16 to the respiratory organs of the patient or to switch a direction to suction the air from the respiratory organs of the patient.

Also, the direction-switching valve unit 30 includes a rotator 31 composed of a pair of partition walls 31a configured to selectively open a communication port 15 formed around the first accommodation part 11 and a reversible motor 32 connected to a shaft of the rotator 31.

Also, a flow path may be formed along the rotation of the rotator 31 through a passage 31b between the partition walls 31a.

That is, the rotator 31 of the direction-switching valve unit 30 rotates to selectively open or close the communication port 15 formed around the first accommodation part 11 and performs a function of inducing a cough, and this is accomplished by performing an inhalation mode, an exhalation mode, and a pause mode largely in one cycle.

Also, for the convenience of explanation, the communication ports 15 will be described separately as a first communication port 15a at a 270° position, a second communication port 15b at a 0° position, and a third communication port 15c at a 90° position.

The direction-switching valve unit 30 is provided with a sensing means 33 configured to control the driving of the reversible motor 32 while sensing the rotation of the rotator 31. Here, as an example, the sensing means 33 may include a disc 33a coupled to an upper shaft of the reversible motor 32 and having marks formed at regular intervals along a rim thereof, and a sensor 33b installed at an upper side of the reversible motor 32 and sensing the marks of the disc 33a.

Here, the marks of the disc 33a may be mode sensing grooves S which may be sensed by the sensor. Here, the mode sensing grooves S may be formed at intervals of 45°, be composed of three grooves of an inhalation mode IN, a pause mode PA and an exhalation mode EX, and sense the mode in which the rotator 31 is rotated to output a signal to a controller (not shown).

The high-frequency oscillation wave generation means 40 is coupled to the second accommodation part 12 and generates a high-frequency oscillation wave when supplying the air introduced from the external air flow port 16 to the respiratory organs or suctioning the air from the respiratory organs.

Also, the high-frequency oscillation wave generation means 40 includes: a fixed body 41 composed of an inner cylinder 41a in which a flow path F is formed on a central axis and a through hole h is formed in a horizontal direction, and an outer cylinder 41b formed on an outer side of the inner cylinder 41a and having an inner circumferential surface provided with a magnet 41c; and a moving body 42 installed between the inner cylinder 41a and the magnet 41c and having a through hole h formed in the horizontal direction and a coil 42a wound around an outer circumferential surface so that the moving body 42 moves longitudinally by the supply of current.

Further, the present invention removes a position sensing magnet provided on an upper part of the moving body 42 and a center position sensing sensor provided on an outside of the magnet as in the related art, and the oscillation and amplitude are controlled by the moving body 42 during inhalation and exhalation and the position of the moving body 42 is controlled by feedback of a pressure of the air flow path.

The operation of the portable in-exsufflator 100 using the high-frequency oscillation wave of the present invention, which is constituted by the above-described configuration, will be described in detail.

Prior to a specific description of the action, the following terms are summarized as follows.

The inhalation mode is an inhalation state in which a patient breathes in, but in the present invention, is described as a state in which the in-exsufflator 100 as the main body uses an air pressure generated in the air pressure generation unit 20 to supply air to the respiratory organs of the patient through the direction-switching valve unit 30 and the high-frequency oscillation wave generation means 40.

The pause mode is an apnea state in which a patient stops breathing, but in the present invention, is described as a state in which the in-exsufflator 100 as the main body stops supplying air to or suctioning air from the patient's respiratory organs by blocking an air pressure suctioned or discharged by the air pressure generation unit 20 by the direction-switching valve unit 30.

The exhalation mode is an exhalation state in which a patient exhales, but in the present invention, is described as a state in which the in-exsufflator 100 as the main body uses an air pressure generated in the air pressure generation unit 20 to discharge air from the respiratory organs of the patient through the direction-switching valve unit 30 and the high-frequency oscillation wave generation means 40.

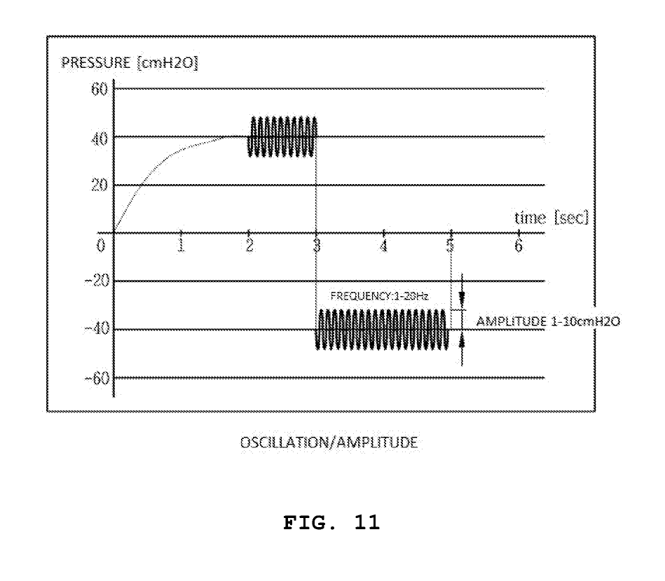

FIG. 11 is a graph illustrating oscillation waves of the portable in-exsufflator using a high-frequency oscillation wave according to the present invention, and referring to the graph, it can be confirmed that an oscillation wave is generated in the inhalation mode and the exhalation mode.

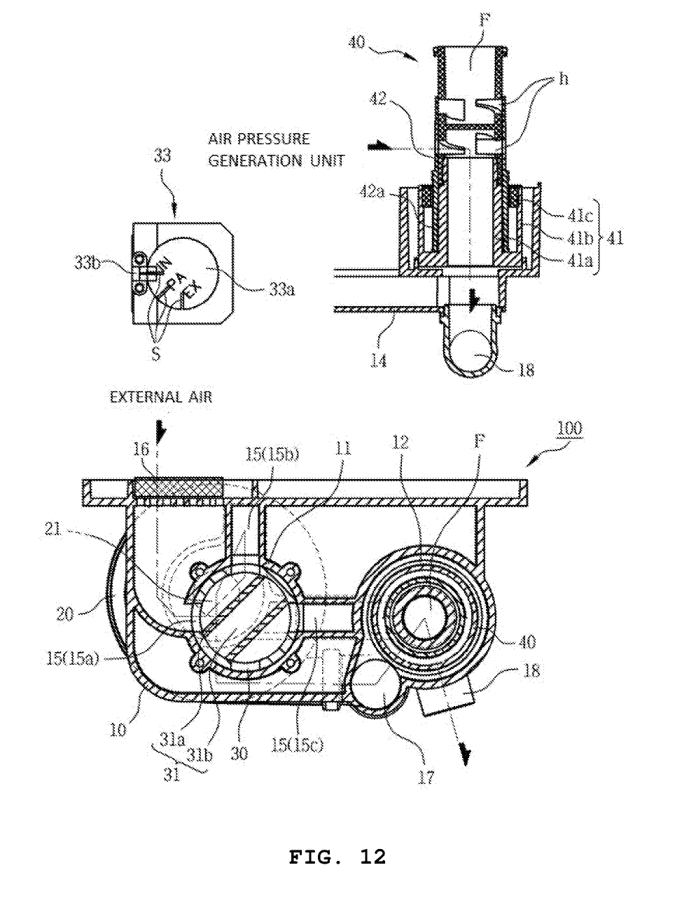

FIG. 12 is a view illustrating an inhalation mode of the portable in-exsufflator using a high-frequency oscillation wave according to the present invention.

For reference, a cross-sectional view of the manifold shows the flow of air, indicates that the portable in-exsufflator is in the inhalation mode IN, as the sensing means, and shows the flow of air as the high-frequency oscillation wave generation means.

First, when power is turned on to use the in-exsufflator 100 and the air pressure generation unit 20 is operated in a state in which a pressure is set, the air pressure generation unit 20 is driven at a rotational speed corresponding to the set pressure, and a mode is manually or automatically set.

In the inhalation mode IN, the direction-switching valve unit is placed in the inhalation state, which causes the rotator 31 of the direction-switching valve unit 30 to be inclined at an angle of 45° in the clockwise direction, so that the first communication port 15a and the second communication port 15b are opened while the third communication port 15c is closed.

The external air flowing through the external air flow port 16 passes through the first and second communication ports 15a and 15b in a state in which the rotator 31 is inclined by 45° in the clockwise direction, and flows into the air suction port 21 of the air pressure generation unit 20 connected to the inhalation chamber 13 and then passes through the air discharge port 22 due to the second communication port 15b vertically communicating with the inhalation chamber 13.

The external air passed through the air discharge port 22 is moved to the air supply chamber 17 communicating with the second accommodation part 12 and then passes through the high-frequency oscillation wave generation means 40 provided in the second accommodation part 12 to supply air to the respiratory organs of the patient through the respiratory flow port 17.

Here, the moving body 42 of the high-frequency oscillation wave generation means 40 is positioned at a lower part, is moved to an upper part by the supply of the current until the set pressure is equal to the − amplitude pressure, and is then moved to a lower part, when the set pressure reaches the amplitude pressure, until the set pressure is equal to the + amplitude pressure. When the operation is operated in accordance with a set frequency, the same result as the graph of FIG. 11 may be obtained (the pressure is PID control).

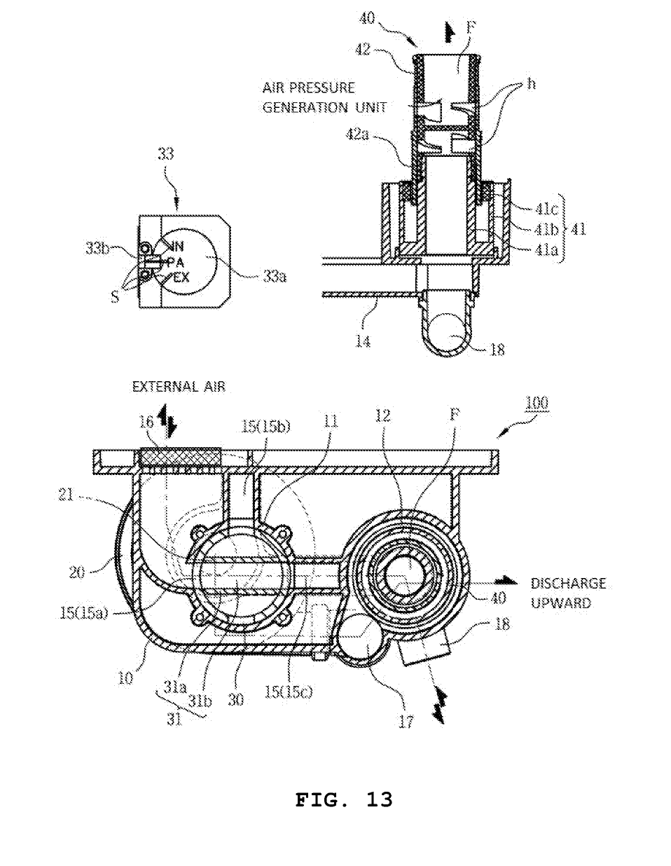

FIG. 13 is a view illustrating a pause mode of the portable in-exsufflator using a high-frequency oscillation wave according to the present invention.

For reference, the cross-sectional view of the manifold shows that the flow of air and indicates that the portable in-exsufflator is in the pause mode PA, as the sensing means.

In the pause mode PA, the direction-switching valve unit is placed in the pause state, which causes the rotator 31 of the direction-switching valve unit 30 to be inclined at an angle of 90° in the clockwise direction, so that the first communication port 15a and the third communication port 15c are opened while the second communication port 15b is closed. Accordingly, the breathing of the patient flowing in and out of the respiratory flow port 18 is moved upward to the third communication port 15c through the exhalation chamber 14. Thereafter, the breathing is performed under atmospheric pressure conditions by the external air flow port 16 communicating with the first communication port 15a through the passage between the partition walls 31a of the rotator 31 of the direction-switching valve unit to provide a comfortable resting function to the patient.

The air pressure generation unit 20 is continuously operated and the air introduced into the air suction port 21 flows into the air discharge port 22 and then passes through the air chamber to be discharged to the upper part through the flow path F formed in the moving body 42 of the high-frequency oscillation wave generation means 40.

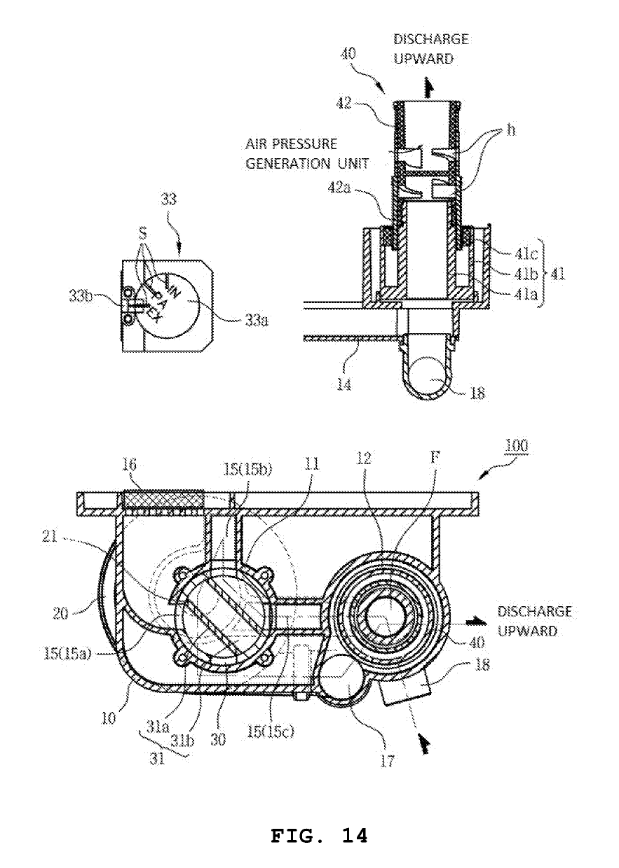

FIG. 14 is a view illustrating an exhalation mode of the portable in-exsufflator using a high-frequency oscillation wave according to the present invention.

For reference, the cross-sectional view of the manifold shows that the flow of air, indicates that the portable in-exsufflator is in the pause mode PA, as the sensing means, and shows the flow of air as the high-frequency oscillation wave generation means.

In the exhalation mode EX, the direction-switching valve unit is placed in the exhalation state, which causes the rotator 31 of the direction-switching valve unit 30 to be inclined at an angle of 45° in the counterclockwise direction, so that the second communication port 15b and the third communication port 15c are opened while the first communication port 15a is closed.

In this state, the air exhaled from the respiratory organs of the patient passes through the exhalation chamber 14 in the respiratory flow port 18 and is introduced into the air suction port 21 of the air pressure generation unit through the third communication port 15c and the second communication port 15b. The introduced air passes through an air supply chamber 17 through the air discharge port 22, and passes through the through hole h of the high-frequency oscillation wave generation means 40 to be discharged to the upper part through the central flow path F.

Here, the moving body 42 of the high-frequency oscillation wave generation means 40 is positioned at the upper part, is moved downward to the lower portion by the supply of the current until the set pressure is equal to the − amplitude pressure, and is then moved upward to the upper part, when the set pressure reaches the amplitude pressure, until the set pressure is equal to the + amplitude pressure. When the operation is operated in accordance with the set frequency, the same result as the graph of FIG. 11 may be obtained (pressure is PID control).

The present invention is described with reference to an embodiment shown in the accompanying drawings. However, it will be understood that various modifications and other embodiments are possible by those skilled in the art.

### DESCRIPTION OF SYMBOLS

100: PORTABLE IN-EXSUFFLATOR
10: MANIFOLD
11: FIRST ACCOMMODATION PART
12: SECOND ACCOMMODATION PART
13: INHALATION CHAMBER
14: EXHALATION CHAMBER
15: COMMUNICATION PORT
15a: FIRST COMMUNICATION PORT
15b: SECOND COMMUNICATION PORT
15c: THRID COMMUNICATION PORT
16: AIR SUPPLY CHAMBER
17: RESPIRATORY FLOW PORT
18: EXTERNAL AIR FLOW PORT
20: AIR PRESSURE GENERATION UNIT
21: AIR SUCTION PORT
22: AIR DISCHARGE PORT
30: DIRECTION-SWITCHING VALVE UNIT
31: ROTATING BODY
31a: PARTITION WALL
31b: PASSAGE
32: REVERSIBLE MOTOR
33: SENSING MEANS
33a: DISC
S: MODE SENSING GROOVE
33b: SENSOR
40: HIGH-FREQUENCY OSCILLATION WAVE GENERATION MEANS
41: FIXED BODY
41a: INNER CYLINDER
F: FLOW PATH
h: THROUGH HOLE
41b: OUTER CYLINDER
41c: MAGNET
42: MOVING BODY
42a: COIL

What is claimed is:

1. A portable in-exsufflator comprising:
a manifold having an upper part in which a first accommodation part and a second accommodation part are formed, and a lower part in which an inhalation chamber and an exhalation chamber are formed;
an air pressure generation unit configured to suction and discharge air through a fan rotating by a rotational force transmitted from a motor, and having an air suction port connected to the inhalation chamber and an air discharge port connected to the second accommodation part;
a direction-switching valve unit coupled to the first accommodation part, and configured to switch a direction so as to allow external air to flow therein and be supplied to respiratory organs or to suction the air from the respiratory organs by using air pressure generated from the air pressure generation unit; and
a high-frequency oscillation wave generation means coupled to the second accommodation part, and configured to generate a high-frequency oscillation wave when external air having flowed therein is supplied to the respiratory organs or the air is suctioned from the respiratory organs,
wherein a plurality of communication ports are formed around the first accommodation part of the manifold such that each of the communication ports communicates with an external air flow port, the inhalation chamber and the exhalation chamber.

2. The portable in-exsufflator of claim 1, wherein the communication port communicating with the inhalation chamber and the exhalation chamber communicates in a vertical direction

3. The portable in-exsufflator of claim 1, wherein the second accommodation part has one side at which an air supply chamber is formed so as to communicate therewith, and a discharge port of the air pressure generation unit is connected to the supply chamber.

4. The portable in-exsufflator of claim 1, wherein the direction-switching valve unit comprises a rotator composed of a pair of partition walls configured to selectively open a communication path formed around the first accommodation part and a reversible motor connected to a shaft of the rotator.

5. The portable in-exsufflator of claim 1, wherein the high-frequency oscillation wave generation means comprises:
a fixed body composed of an inner cylinder in which a flow path is formed on a central axis and a through hole is formed in a horizontal direction, and an outer cylinder formed on an outer side of the inner cylinder and having an inner circumferential surface provided with a magnet; and
a moving body installed between the inner cylinder and the magnet and having a through hole formed in the horizontal direction and a coil wound around an outer circumferential surface so that the moving body moves longitudinally by the supply of current.

6. The portable in-exsufflator of claim 5, wherein an oscillation and amplitude are controlled by the moving body during inhalation and exhalation, and a position of the moving body is controlled by feedback of a pressure of an air flow path.
